(19) United States
(12) Patent Application Publication
(10) Pub. No.: US 2018/0148416 A1
(43) Pub. Date: May 31, 2018

(54) INDUSTRIAL PROCESS FOR THE PREPARATION OF ENZALUTAMIDE

(71) Applicant: Olon S.P.A., Rodano (MI) (IT)

(72) Inventors: Samuele Frigoli, Rodano (MI) (IT); Davide Longoni, Rodano (MI) (IT); Marco Alpegiani, Rodano (MI) (IT)

(73) Assignee: Olon S.P.A., Rodano (MI) (IT)

(21) Appl. No.: 15/576,298

(22) PCT Filed: May 24, 2016
(86) PCT No.: PCT/EP2016/061689

§ 371 Date: Nov. 22, 2017

(30) Foreign Application Priority Data
May 28, 2015 (IT) 102015000019007

(51) Int. Cl. C07D 233/86

(52) U.S. Cl. CPC C07D 233/86

(57) ABSTRACT

Disclosed is an efficient method of synthesising Enzalutamide, which comprises the cyclisation reaction of isothiocyanate 1 with acid 3 pre-treated with a silylating agent, or reacting 1 and 3 in the presence of a silylating agent.

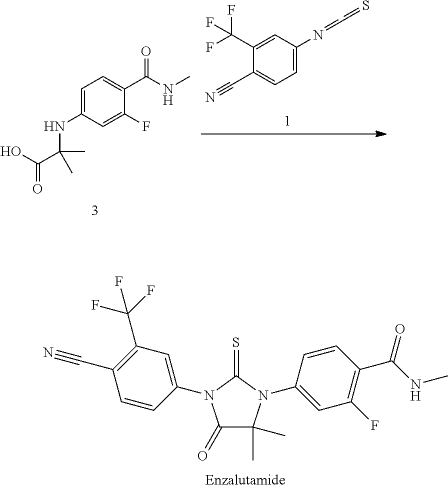

### OBJECT OF THE INVENTION

The object of the invention is a process for the preparation of the active ingredient Enzalutamide.

### PRIOR ART

Non-steroidal androgen receptor (AR) inhibitors, such as bicalutamide, nilutamide and flutamide, have been used for decades to treat prostate cancer, and constituted the gold standard for systematic treatment of castration-resistant prostate cancer until the introduction of new drugs with a different action mechanism, such as docetaxel and abiraterone. Renewed interest in antiandrogens was generated by the discovery of Enzalutamide, a novel inhibitor of ARs adapted to cells that grow in a low-testosterone environment (as in the case of prostate cancer with castration).

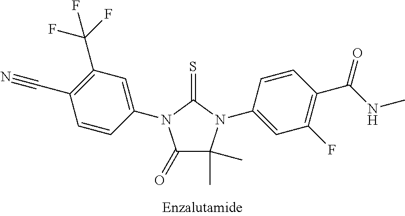 [SMILES: CNC(=O)c1ccc(N2C(=S)N(c3ccc(C#N)c(C(F)(F)F)c3)C(=O)C2(C)C)cc1F]

Enzalutamide, the chemical name of which is 4-{3-[4-cyano-3-(trifluoromethyl)phenyl]-5,5-dimethyl-1-oxo-2-thioxoimidazolidin-1-yl}-2-fluoro-N-methylbenzamide, is the active ingredient of the medicament Xtandi, indicated for the treatment of adult males suffering from metastatic castration-resistant prostate cancer. It is better tolerated and more effective than the first antiandrogens, significantly contributing to an improvement in the most important oncological endpoints, including quality of life and global survival.

Enzalutamide is claimed in WO2006124118, WO2007127010 EP01893196B1, U.S. Pat. No. 7,709,517B2 and U.S. Pat. No. 8,183,274B2; the last step of the preparation method described (Scheme 1) is microwave-assisted cycloaddition of isothiocyanate 1 with cyano derivative 2. The reaction takes place with low yields, and chromatographic purification is required; moreover, the preparation of a cyanoalkylamine derivative such as 2 requires the use of cyanides or cyanohydrin.

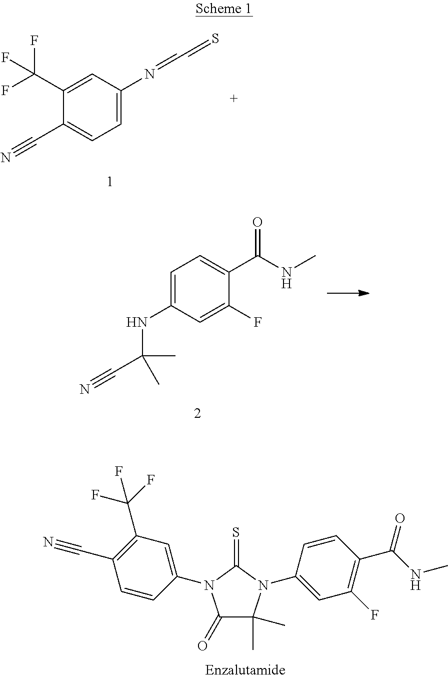 [SMILES: N#Cc1ccc(N=CC=S)cc1C(F)(F)F ; "+" ; CNC(=O)c1ccc(NC(C)(C)C#N)cc1F ; CNC(=O)c1ccc(N2C(=S)N(c3ccc(C#N)c(C(F)(F)F)c3)C(=O)C2(C)C)cc1F]

A more efficient process for the preparation of Enzalutamide, described in WO2011106570, involves cyclisation of isothiocyanate 1 with methyl ester 4, or a homologue thereof, obtained by esterification of acid 3 (Scheme 2).

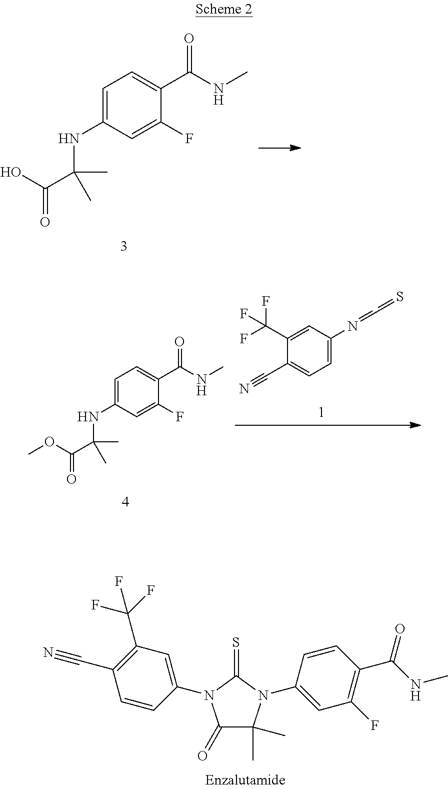

The synthesis of Enzalutamide directly from acid 3, which is particularly attractive and advantageous because isolation of ester 4 is avoided, does not appear to have been described for the afore-mentioned API, although there are precedents in the synthesis of 2,4-imidazolidindiones and 2-thioxo-4-imidazolidinones, for example as described in WO02081453 and WO2006028226.

We therefore tested the cyclisation of acid 3, or a salt thereof with a tertiary amine, with isothiocyanate 1, observing the formation of Enzalutamide with modest yields and the need for complex purifications to obtain an API of acceptable quality.

We surprisingly found that said cyclisation takes place with high yields and quality, and under milder conditions, if the acid is pre-treated with a silylating agent or the reaction is conducted in the presence of a silylating agent.

### DESCRIPTION OF THE INVENTION

“Silylation” means substitution of one or more active hydrogens of an organic compound with a trisubstituted silyl group (such as an R₃Si— group). Organic compounds with active hydrogens are generally characterized by the presence of a —OH group, like carboxylic acids, alcohols or phenols, or a —NH group, like amines, amides or ureas, or a —SH group, like thiols; and the silylating agent is usually a trialkylsilyl halide or an N-derivative or O-derivative trialkylsilyl compound such as N-silylamides, N,O-bis(silyl)amides, N,O-bis(silyl)carbamates, N,N′-bis(silyl)ureas or N,O-bis(silyl)sulphamates.

We have found that Enzalutamide can be advantageously synthesised if acid 3 is treated with a silylating agent and then reacted with isothiocyanate 1. The result is Enzalutamide with very high conversion and yields; the isolation of the active ingredient is greatly facilitated, and the quality is very high.

Types of silylating agents and silylation methodologies are described in detail in the literature. See, for example: Peter G. M. W., Greene's Protective Groups in Organic Synthesis, 5th Edition, 2014; Pape, P. G., “Silylating Agents”, Kirk-Othmer Encyclopedia of Chemical Technology, 2006; Kashutina M. V., Russ. Chem. Rev. 44, 733 (1975); Roth, C. A., Industrial & Engineering Chemistry Product Research and Development 11, 134 (1972), and the references reported therein.

The preferred silyl groups are trialkylsilyls, such as trimethylsilyl, triethylsilyl, tri-n-propylsilyl, methyldiethylsilyl, dimethylethylsilyl, phenyldimethylsilyl, tert-butyldimethylsilyl, tert-butyldiphenylsilyl and triphenylsilyl.

A particularly preferred silyl group is trimethylsilyl, due to its characteristics including mild introduction and removal conditions, and the availability of numerous low-cost trimethylsilylating agents on the market.

Examples of trimethylsilylating agents are chlorotrimethylsilane, hexamethyldisilazane, N,O-bis(trimethylsilyl)acetamide, N,O-bis(trimethylsilyl)carbamate, N,N′-bis(trimethylsilyl)urea, 3-trimethylsilyl-2-oxazolidinone, N-(trimethylsilyl)acetamide, N-methyl-N-trimethylsilylacetamide, N-trimethylsilylimidazole, 1-methoxy-2-methyl-1-trimethyloxypropene, (isopropenyloxy)trimethylsilane, N,O-bis(trimethylsilyl)sulphamate, allyltrimethylsilane, and mixtures thereof.

Silylation is typically performed in aprotic solvent or mixtures of aprotic solvents, and can be facilitated in polar solvents. The process may also require the presence of an acid catalyst such as trifluoroacetic acid, p-toluenesulphonic acid or sulphuric acid, a salt such as ammonium sulphate or pyridinium p-toluenesulphonate, or a basic catalyst such as pyridine, which can also be used as solvent or co-solvent. Chlorotrimethylsilane can be used as catalyst together with another silylating agent.

If an acid by-product forms from the silylation reaction, the use of an acid acceptor may be indicated, and the salt formed can be removed by filtration: one example is the use of chlorotrimethylsilane in the presence of a tertiary amine, and the removal by filtration of the tertiary amine hydrochloride deriving from the silylation reaction.

In other cases, the by-product formed by the silylation reaction can easily be removed, either because it is poorly soluble, such as urea in the case of N,N′-bis(trimethylsilyl)urea, or because it is volatile, such as ammonia in the case of hexamethyldisilazane.

Often, however, it is unnecessary to remove the by-product of the silylation reaction from the mixture; the subsequent reaction can be performed immediately, with no need for filtration, distillation, concentration under vacuum, change of solvent, isolation or other operations.

The action of the silylating agent on acid 3 can be assumed to give rise to a derivative wherein the carboxyl functionality is primarily protected, but other active hydrogens present in the molecule may also be at least partly silylated.

Condensation with isothiocyanate 1 to give Enzalutamide can be effected a) after subjecting acid 3 to the action of the silylating agent, or b) the condensation reaction between 1 and 3 can be conducted in the presence of the silylating agent.

The condensation reaction is typically effected in an aprotic organic solvent or a mixture of aprotic solvents, selected from an ester such as ethyl acetate, propyl acetate, isopropyl acetate or butyl acetate, an ether such as tetrahydrofuran, methyltetrahydrofuran, dioxane, tert-butyl methyl ether or cyclopentyl methyl ether, an amide such as N,N-dimethylformamide, N,N-dimethylacetamide or N-methyl pyrrolidone, an aromatic hydrocarbon such as toluene or xylene, or another solvent such as methylene chloride, acetonitrile, dimethylsulphoxide, sulfolane or N,N′-dimethyl-propylene urea. The reaction temperature typically ranges from +20° C. to +150° C., preferably from +40 to +120° C.; the reaction time ranges from 1 hour to 60 hours, preferably from 2 hours to 40 hours.

The molar ratio of species 3 to isothiocyanate 1 generally ranges from 1:1 to 1:4, preferably from 1:1.1 to 1:2.5. With reference to the carboxyl functionality of acid 3, the molar equivalents of the silylating agent preferably range from 1 to 4.

The conversion of acid 3 generally exceeds 90%, and the molar yield of Enzalutamide vs. species 3 typically exceeds 70%.

The isolation of Enzalutamide typically does not require chromatographic purifications. It can comprise treatment with a protic solvent, for example an alcohol such as methanol, ethanol or propanol, or with a neutral, acid or basic aqueous solution. The isolation can then be performed by one of the classic methods, such as precipitation of the crude product by adding anti-solvent to the reaction mixture; or dilution with a suitable solvent, optional washing of the organic solution with aqueous solutions, and obtaining the crude product by concentrating the organic phase.

The quality of the crude product can then be enhanced by treating it with solvent (slurry), by treating a solution thereof with decolourising charcoal or another absorbent material, or by crystallisation.

The products of formula 1 and 3 are known products, or can be prepared from known products by known methods.

Isothiocyanate 1 is easily obtained by reaction from amine 4, used to prepare other active ingredients such as bicalutamide, by reaction with thiocarbonyl dichloride (Scheme 3) [for preparation examples see, for example, WO 2006133567; Chemical & Pharmaceutical Bulletin 56, 1555 (2008)].

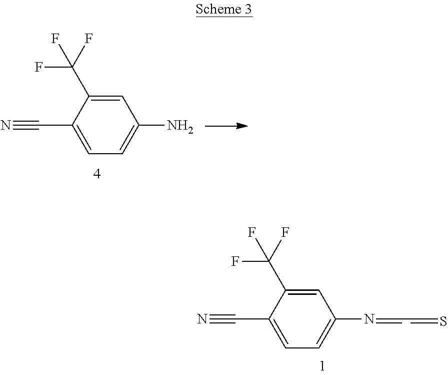 [SMILES: N#Cc1ccc(N)cc1C(F)(F)F ; N#Cc1ccc(N=CC=S)cc1C(F)(F)F]

Acid 3 can be prepared, for example, by analogy with the general methods described in the literature, starting with aniline 5 (Scheme 4) by alkylation with bromoisobutyric acid or an ester thereof and subsequent hydrolysis [see, for example, WO02081453, WO 2011128251, J. Med. Chem. 54, 6254 (2011)], or using 5 as nucleophilic partner in the Bargellini reaction [see, for example, ARKIVOC 2012 Part (ii) 24-40; Tetrahedron Letters 50, 2497 (2009)], wherein chloretone (1,1,1-trichloro-2-methyl-2-propanol) can be used “as is” or obtained in situ from acetone and chloroform.

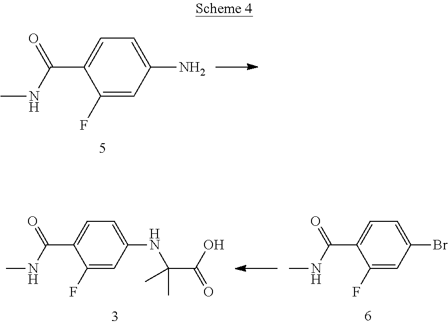 [SMILES: CNC(=O)c1ccc(N)cc1F ; CNC(=O)c1ccc(NC(C)(C)C(=O)O)cc1F ; CNC(=O)c1ccc(Br)cc1F]

Alternatively, acid 3 can be obtained from bromo derivative 6 (Scheme 4) by nucleophilic substitution with 2-methyl alanine [see, for example, WO2006028226, Tetrahedron Letters 50, 5159 (2009); Bioorganic & Medicinal Chemistry 14, 6789 (2006)].

The invention will now be illustrated by the following examples.

### Example 1

### Synthesis of Enzalutamide in the presence of N,O-bis(trimethylsilyl)acetamide (BSA)

BSA (14 ml) is added to a suspension of 2-(3-fluoro-4-methylcarbamoyl-phenylamino)-2-methyl-propionic acid (14 g) in DMSO (15 ml) and isopropyl acetate (30 ml), and stirred at room temperature to obtain a solution. 4-isothiocyanato-2-trifluoromethyl-benzonitrile (20 g) is added, and the resulting mixture is heated at 55-60° C. for about 24 hours. The reaction mixture is cooled to 25° C., and isopropyl acetate, isopropyl alcohol (IPA) and water are added. The organic phase is separated and concentrated under vacuum, and the residue is crystallised from IPA. The wet solid (about 25 g) is taken up in DCM (160 ml), and the resulting solution is treated with CPL charcoal (1 g) and filtered through dicalite. The filtrate is concentrated and the residue is crystallised from n-heptane/ethyl acetate. The product is dried under vacuum at 55° C. for 20 hours. 20 g of Enzalutamide is obtained.

### Example 2

### Synthesis of Enzalutamide in the Absence of BSA

A mixture of 2-(3-fluoro-4-methylcarbamoyl-phenylamino)-2-methyl-propionic acid (14 g) and 4-isothiocyanato-2-trifluoromethyl-benzonitrile (20 g) in DMSO (15 ml) and isopropyl acetate (30 ml) is heated at 70-75° C. for 24 hours. The reaction mixture is cooled to 25° C., and isopropyl acetate, IPA and water are added. The insoluble material is filtered off, and the organic phase is separated and concentrated under vacuum. Chromatographic purification (silica gel, eluent: n-heptane/ethyl acetate) is required to isolate Enzalutamide. 7 g of Enzalutamide is obtained from the eluate after concentration under vacuum, filtration and drying.

### Example 3

### Synthesis of Enzalutamide in the Presence of Tertiary Amine

A mixture of 2-(3-fluoro-4-methylcarbamoyl-phenylamino)-2-methyl-propionic acid (14 g), TEA (8 ml) and 4-isothiocyanato-2-trifluoromethyl-benzonitrile (20 g) in DMSO (15 ml) and isopropyl acetate (30 ml) is heated at 86-90° C. for 24 hours. The reaction mixture is cooled to 25° C., and isopropyl acetate, IPA and water are added. The insoluble material is removed by filtration, and the organic phase is separated and concentrated under vacuum. Chromatographic purification (silica gel, eluent: n-heptane/ethyl acetate) is required to isolate Enzalutamide. 3 g of Enzalutamide is obtained from the eluate after concentration under vacuum, filtration and drying.

1. A process for the preparation of 4-{3-[4-cyano-3-(trifluoromethyl)phenyl]-5,5-dimethyl-1-oxo-2-thioxoimidazolidin-1-yl}-2-fluoro-N-methylbenzamide (enzalutamide) by means of condensation of a compound of formula 3 with a compound of formula 1 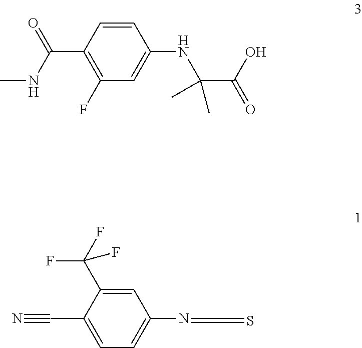 [SMILES: CNC(=O)c1ccc(NC(C)(C)C(=O)O)cc1F ; N#Cc1ccc(N=C=S)cc1C(F)(F)F]
wherein:
a. said condensation is carried out after subjecting 3 to the action of a silylating agent; or
b. said condensation is carried out in the presence of a silylating agent.

2. The process according to claim 1 wherein the silylating agent contains a silyl group selected from trimethylsilyl, triethylsilyl, tri-n-propylsilyl, methyldiethylsilyl, dimethylethylsilyl, phenyldimethylsilyl, tert-butyldimethylsilyl, tert-butyldiphenylsilyl and triphenylsilyl.

3. The process according to claim 2 wherein the silylating agent contains a trimethylsilyl group.

4. The process according to claim 3 wherein the silylating agent is selected from chlorotrimethylsilane, hexamethyldisilazane, N,O-bis(trimethylsilyl)acetamide, N,O-bis(trimethylsilyl)carbamate, N,N′-bis(trimethylsilyl))urea, 3-(trimethylsilyl)-2-oxazolidinone, N-(trimethylsilyl)acetamide, N-methyl-N-(trimethylsilyl)acetamide, N-(trimethylsilyl)imidazole, 1-methoxy-2-methyl-1-trimethylsilyloxypropene, (isopropenyloxy)trimethylsilane, N,O-bis(trimethylsilyl)sulphamate, allyltrimethylsilane, and mixtures thereof.

5. The process according to claim 1 wherein the molar ratio of compound 3 to compound 1 ranges from 1:1 to 1:4.

6. The process according to claim 1 wherein, with reference to the carboxyl functionality of 3, the molar equivalents of the silylating agent range from 1 to 4.
